(19) United States
(12) Patent Application Publication
(10) Pub. No.: US 2024/0207817 A1
(43) Pub. Date: Jun. 27, 2024

(54) AGAROSE-CELLULOSE NANOCOMPOSITE POROUS GEL MICROSPHERE, PREPARATION METHOD, AND APPLICATION

(71) Applicant: Hangzhou NeuroPeptide Biological Science and Technology Incorporation, Ltd. (NUPTEC), Zhejiang (CN)

(72) Inventors: Xianpeng YANG, Zhejiang (CN); Yongchang Qian, Zhejiang (CN); Wenjun Ye, Zhejiang (CN)

(73) Assignee: Hangzhou NeuroPeptide Biological Science and Technology Incorporation, Ltd. (NUPTEC), Zhejiang (CN)

(21) Appl. No.: 18/190,151

(22) Filed: Mar. 27, 2023

(30) Foreign Application Priority Data
Dec. 26, 2022 (CN) 202211680006.9

(51) Int. Cl. B01J 20/285; B01D 15/38; B01J 20/28; B01J 20/291; B01J 20/30; C07K 1/22; C08J 9/00; C08J 9/20; C08J 9/36

(52) U.S. Cl. CPC B01J 20/285; B01D 15/3828; B01J 20/28019; B01J 20/291; B01J 20/305; C07K 1/22; C08J 9/0061; C08J 9/20; C08J 9/36; B01J 2220/445; B01J 2220/54; B01J 2220/58; C08J 2201/026; C08J 2203/22; C08J 2301/02; C08J 2405/00

(57) ABSTRACT

The disclosure provides an agarose-cellulose nanocomposite porous gel microsphere, a preparation method, and an application. In the disclosure, agarose and nanocellulose are compounded to form a unique network structure by using an industrially scalable method, that is, a reversed-phase emulsification method. The maximum flow rate and pressure resistance of the porous gel microsphere are significantly improved. In addition, after the composite porous gel microsphere is modified with a specific ligand, the dynamic binding capacity of the separation target is improved, and the modified composite porous gel microsphere can be used for large-scale separation and purification of biological macromolecules. The disclosure adapts to the development trend of high rigidity, high flow rate, and high loading capacity of the chromatography medium, and is expected to be used as the next-generation chromatography medium with this performance.

### CROSS REFERENCE TO RELATED APPLICATION

This application claims the priority benefit of to China application serial no. 202211680006.9, filed on Dec. 26, 2022. The entirety of the above-mentioned patent application is hereby incorporated by reference herein and made a part of this specification.

### BACKGROUND

### Technical Field

The disclosure relates to the technical field of microsphere preparation, and in particular, relates to an agarose-cellulose nanocomposite porous gel microsphere, a preparation method, and an application.

### Description of Related Art

Chromatography is one of the most effective methods for the separation and purification of biomacromolecules such as monoclonal antibodies and nucleic acids. The chromatography medium is the core basic material of biological separation. Porous gel microspheres are the most widely used chromatography media for bioseparation in industry, and are mainly divided into natural polymers and synthetic polymers according to their sources. Chromatography media based on natural polymers such as agarose and cellulose have occupied a dominant position due to their low extractables and high biological safety. For instance, agarose porous gel microsphere Sepharose® has been enduring since its launch in 1966. On the other hand, the rapid development of biotechnology has continuously increased the demand for chromatography media, and the requirements have also been continuously improved. Compared with synthetic polymers, natural polymer-based chromatography media have the disadvantage of poor mechanical properties. The conventional single natural polymer porous gel microspheres are difficult to meet the requirements of high rigidity, high flow rate, and high loading at the same time.

The advantages of agarose for preparing porous gel microspheres are good water solubility, suitable gelation temperature, and high gel strength. Compared with the agarose chromatography medium, the raw material of cellulose chromatography medium is affordable, and the cellulose molecular chain can form a crystalline structure, so the skeleton strength is higher. Using agarose and cellulose to form composite porous gel microspheres may combine the advantages of both to improve the performance of chromatography media.

Chinese patent CN112619612A discloses a method for preparing high-strength cellulose/agarose composite microspheres. In this method, it is necessary to dissolve cellulose and agarose separately with an alkali urea solution under low temperature conditions. It thus can be seen that the process is relatively complicated, the requirements for equipment are high, and the obtained microspheres have poor sphericity and are not suitable for large-scale industrial chromatography. On the other hand, natural cellulose has a multi-level structure, and nanocellulose can be obtained through chemical, physical, biological, or combined methods. Nanocellulose is widely used in nanocomposites as a reinforcing phase, and the design of nanocomposites is also suitable for agarose/cellulose porous gel microspheres. Chinese patent CN111989155A discloses a method for reinforcing agarose microspheres with bifurcated submicron cellulose. The combination of submicron cellulose and agarose enhances the rigidity of low-concentration agarose microspheres. However, the size of the cellulose is excessively large, resulting in mostly ellipsoidal microspheres, which is not conducive to the chromatography effect. Further, submicron cellulose also has the risk of being unable to embed inside the agarose microspheres.

### SUMMARY

The first purpose of the disclosure is to provide a preparation method of an agarose-cellulose nanocomposite porous gel microsphere, aiming to solve the deficiencies in the related art.

For this reason, the abovementioned purpose of the disclosure is achieved through the following technical solutions.

The disclosure provides a preparation method of an agarose-cellulose nanocomposite porous gel microsphere, and preparation method includes the following steps.

In S1, agarose is dissolved in a dispersion of nanocellulose as a water phase.

0.01 to 10 parts by weight of nanocellulose are dispersed in 100 parts by weight of water to form a uniform dispersion, and 0.5-20 parts by weight of agarose is added and heated under stirring until the agarose is completely dissolved.

In S2, the agarose-cellulose nanocomposite porous gel microsphere is prepared through a reversed-phase emulsification method.

The water phase obtained in step S1 is poured into an oil phase heated to 50-90° C., mechanically stirred and emulsified for 10-30 minutes, a rotation speed is adjusted so that the water phase is dispersed into a droplet of a required particle size, the emulsion is cooled at a rate of 2° C. per minute to below 20° C. to gel the droplet of the water phase, and an uncrosslinked agarose-cellulose nanocomposite gel microsphere is obtained after washing.

The oil phase includes a water-immiscible organic solvent and a single or compound emulsifier with an HLB value of 3 to 8.

In S3, the agarose-cellulose nanocomposite porous gel microsphere is cross-linked.

Epichlorohydrin is used to cross-link agarose and cellulose under alkaline conditions to form the agarose-cellulose nanocomposite porous gel microsphere, and an amount of the epichlorohydrin is 1-20% of based on a volume of the microsphere.

While adopting the above technical solutions, the following technical solutions are also adopted or combined in the disclosure.

In a preferred technical solution of the disclosure, the nanocellulose is cellulose nanofibrils or cellulose nanocrystal.

Preferably, the cellulose nanofibrils are cellulose nanofibrils RHEOCRYSTA or cellulose nanofibrils modified by carboxymethylation.

The cellulose nanocrystal is obtained by hydrolysis of microcrystalline cellulose.

In a preferred technical solution of the disclosure, the nanocellulose is present in an aggregated state with a diameter of 2-100 nm and a length of less than 10 μm, is fibril-shaped or rod-shaped, and is not comb-shaped or fork-shaped.

In a preferred technical solution of the disclosure, the diameter of the nanocellulose is preferably 2-50 nm, most preferably 2-20 nm.

In a preferred technical solution of the disclosure, a crystalline region is provided in the nanocellulose, and a surface of the nanocellulose has a molecular chain of cellulose or a molecular chain of cellulose derivative.

In a preferred technical solution of the disclosure, the nanocellulose is able be dispersed in water to form an individual particle or fibrils or form a network structure through physical entanglement and non-covalent interactions.

In a preferred technical solution of the disclosure, in step S1, the agarose is preferably 4 to 6 parts by weight, and the nanocellulose is preferably 0.1 to 1 parts by weight.

In a preferred technical solution of the disclosure, in step S2, the organic solvent in the oil phase is at least one of cyclohexane and liquid paraffin.

The emulsifier in the oil phase is at least one of Span 85, Span 80 and Span 60.

In a preferred technical solution of the disclosure, the oil phase also includes Tween 80 to regulate HLB.

In a preferred technical solution of the disclosure, in step S3, the alkaline condition is achieved by adding sodium hydroxide.

In a preferred technical solution of the disclosure, in step S3, the amount of epichlorohydrin is preferably 5-15% of the volume of the composite gel microsphere.

The second purpose of the disclosure is to provide an agarose-cellulose nanocomposite porous gel microsphere.

For this reason, the abovementioned purpose of the disclosure is achieved through the following technical solutions.

The disclosure further provides an agarose-cellulose nanocomposite porous gel microsphere, and the agarose-cellulose nanocomposite porous gel microsphere is prepared and obtained through the abovementioned preparation method of the agarose-cellulose nanocomposite porous gel microsphere.

While adopting the above technical solutions, the following technical solutions are also adopted or combined in the disclosure.

In a preferred technical solution of the disclosure, a mass of the nanocellulose contained in the agarose-cellulose nanocomposite porous gel microsphere accounts for 0.1-200% of a mass of the agarose.

In a preferred technical solution of the disclosure, the mass of the nanocellulose contained in the agarose-cellulose nanocomposite porous gel microsphere accounts for preferably 1-50% of the mass of the agarose, most preferably 1-20%.

In a preferred technical solution of the disclosure, the agarose-cellulose nanocomposite porous gel microsphere is spherical or approximately spherical, with a diameter of 20-300 μm.

In a preferred technical solution of the disclosure, the diameter of the agarose-cellulose nanocomposite porous gel microsphere is preferably 50-150 μm.

In a preferred technical solution of the disclosure, the agarose-cellulose nanocomposite porous gel microsphere includes an independent agarose network and also includes a semi-interpenetrating network or double network structures formed by nanocellulose and agarose.

In a preferred technical solution of the disclosure, a chemical cross-link is provided between the nanocellulose, a chemical cross-link is provided between the agarose, and a chemical cross-link is also provided between the nanocellulose and the agarose.

The third purpose of the disclosure is to provide a chromatography medium having an agarose-cellulose nanocomposite porous gel microsphere.

For this reason, the abovementioned purpose of the disclosure is achieved through the following technical solutions.

The disclosure further provides a chromatography medium having an agarose-cellulose nanocomposite porous gel microsphere, and the chromatography medium is obtained by modifying the abovementioned agarose-cellulose nanocomposite porous gel microsphere with a ligand.

Another purpose of the disclosure is to provide an application of the abovementioned agarose-cellulose nanocomposite porous gel microsphere for separating and purifying a biological macromolecule.

For this reason, the abovementioned purpose of the disclosure is achieved through the following technical solutions.

According to the abovementioned application of the agarose-cellulose nanocomposite porous gel microsphere for separating and purifying the biological macromolecule, the abovementioned agarose-cellulose nanocomposite porous gel microsphere is modified with a ligand and is used for the separation and purification of biological macromolecules such as proteins and nucleic acids.

The mode of separation and purification of biomacromolecules is not particularly limited, and it can be common chromatography methods such as affinity, hydrophobic interaction, ion exchange, gel filtration, and mixed mode.

The disclosure provides an agarose-cellulose nanocomposite porous gel microsphere, a preparation method, and an application. According to the relevant theory of nanocomposite materials, the effect of using smaller nanocellulose to enhance agarose microspheres is better than that of using submicron cellulose. Further, nanocellulose can be completely contained inside the microsphere more easily, and an agarose/cellulose nanocomposite porous gel microsphere with better sphericity is thus obtained. In the disclosure, agarose and nanocellulose are compounded to form a unique network structure by using an industrially scalable method, that is, a reversed-phase emulsification method. The maximum flow rate and pressure resistance of the porous gel microsphere are significantly improved. In addition, after the composite porous gel microsphere is modified with a specific ligand, the dynamic binding capacity of the separation target is improved, and the modified composite porous gel microsphere can be used for large-scale separation and purification of biological macromolecules. The disclosure adapts to the development trend of high rigidity, high flow rate, and high loading capacity of the chromatography medium, and is expected to be used as the next-generation chromatography medium with this performance.

### BRIEF DESCRIPTION OF THE DRAWINGS

FIG. 1 is a transmission electron microscope image of nanocellulose.

FIG. 2 is a micrograph of agarose-cellulose nanocomposite porous gel microspheres.

FIG. 3 is a particle size distribution graph of the agarose-cellulose nanocomposite porous gel microspheres.

FIG. 4 is a pressure/flow rate characteristic curve of the agarose-cellulose nanocomposite porous gel microspheres.

### DESCRIPTION OF THE EMBODIMENTS

The disclosure will be described in detail below in together with specific embodiments, but the embodiments of the disclosure are not limited thereto.

### I. Description of Raw Materials

Three kinds of nanocellulose raw materials are involved in the embodiments of the disclosure.

The first is RHEOCRYSTA, a cellulose nanofibril produced by Dai-ichi Kogyo Seiyaku of Japan. The hydroxyl group at the 6-position of the cellulose molecular chain on the surface of the nanofibrils was partially oxidized to a carboxyl group, and the concentration of RHEOCRYSTA used was 2.65%.

The second is to use bleached sugarcane pulp available on the market as a raw material. The sugarcane pulp was modified by carboxymethylation according to the method provided by the published literature (Cellulose (2018) 25:5781 to 5789). The modified slurry was dispersed in water, and cellulose nanofibrils were obtained after high-speed shearing with a blender (Philips HR3752) for 30 min, which was recorded as CM-CNF with a concentration of 0.38%.

The third one uses microcrystalline cellulose (Sinopharm Reagent No. 68005761) as raw material. According to the method in the published literature (Colloids Surfaces A: Physicochem. Eng. Aspects 142 (1998) 75 to 82), the microcrystalline cellulose was acid-hydrolyzed to obtain cellulose nanocrystals, which were recorded as CNC, and the concentration was 0.50%.

### II. Test Part

### Example 1

A preparation method of an agarose-cellulose nanocomposite porous gel microsphere includes the following steps.

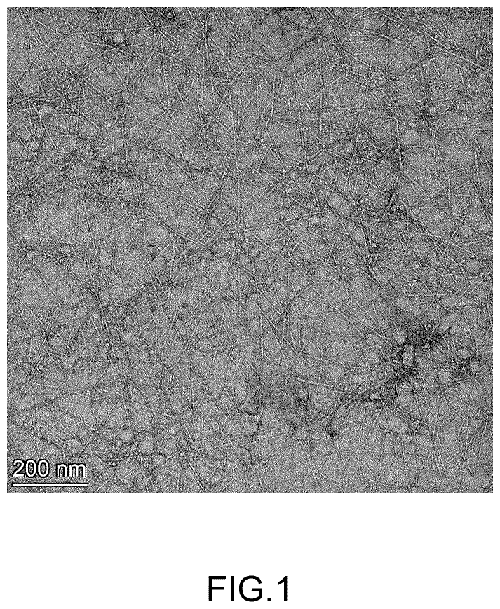

S1: dissolving agarose in a dispersion of cellulose nanoparticles as a water phase:
7.5 g of RHEOCRYSTA with a concentration of 2.65% was weighed, and its transmission electron microscope image is shown in FIG. 1. RHEOCRYSTA was heated to 70° C. by adding 88.5 g of water with stirring, then added 4.0 g of agarose so that the amount of nanocellulose was 5 wt % of the agarose, and heated and stirred at 95° C. to dissolve. After the agarose was completely dissolved, it was kept heating and stirring for 30 min, and it was used as the water phase for later use.

S2: preparing the agarose-cellulose composite porous gel microsphere through a reversed-phase emulsification method:
0.8 g of Tween 80, 7.2 g of Span 80, 40 mL of liquid paraffin, and 160 mL of cyclohexane were added to a 500 mL three-neck round bottom flask, heated and stirred to 50° C., and used as an oil phase for later use. The water phase was added to the stirred oil phase, the emulsification speed was 1,500 rpm, the emulsification temperature was 70° C., and the emulsification time was 20 min. After emulsification, the emulsion was lowered to below 20° C. at a rate of 2° C./min to form gel microspheres, which were washed repeatedly with ethanol and water to obtain 100 mL of gel microspheres.

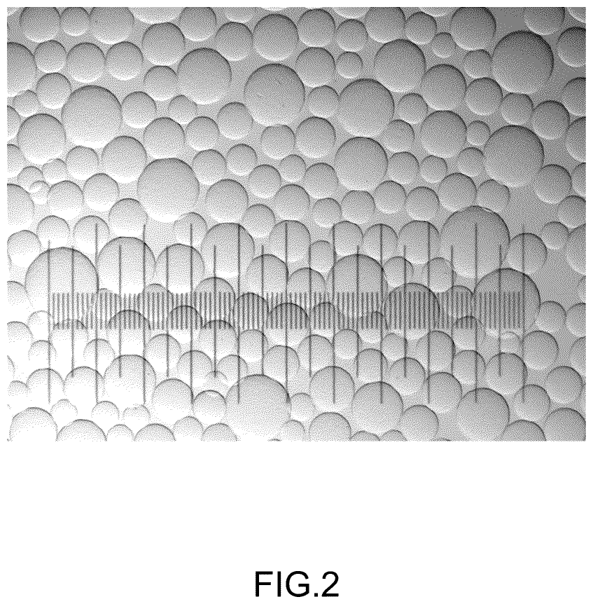

S3: cross-linking of the agarose-cellulose composite porous gel microsphere:
The gel microspheres obtained in step S2 were placed in a 250 mL three-neck round bottom flask, and 75 mL of 2.5 mol/L Na₂SO₄ solution was added, and stirred at 40° C. for 40 min. 2.0 ml of 45 wt % NaOH solution and 0.2 g of NaBH₄ were added and stirred for 30 min. The temperature was raised to 50° C., and 8.5 mL of 45 wt % NaOH solution and 10 mL of epichlorohydrin were added dropwise within 3 hours. After the dropwise addition, the temperature was raised to 60° C., and the reaction was continued for 16 h. The agarose-cellulose nanocomposite porous gel microspheres after sieving were washed with a large amount of pure water to neutrality, as shown in FIG. 2. FIG. 2 is a micrograph of agarose-cellulose nanocomposite porous gel microspheres, and each division in a scale bar in the figure is 10 μm.

### Particle Size Test of Agarose-Cellulose Nanocomposite Porous Gel Microspheres

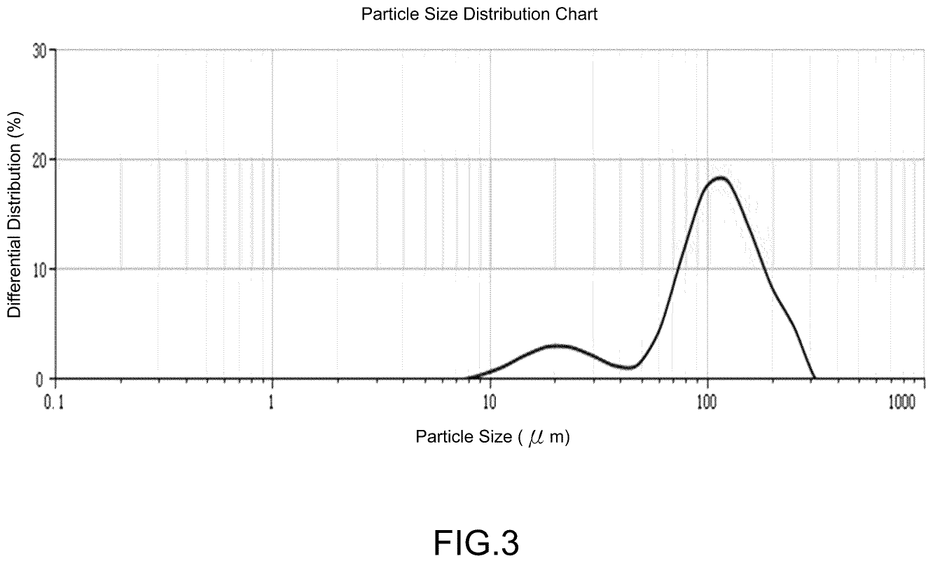

The obtained cross-linked agarose-cellulose nanocomposite porous gel microspheres were tested with a LS-POP (9) laser particle size analyzer, and the average particle size was 109 μm. The particle size distribution graph is shown in FIG. 3.

Pressure/Flow Rate Test of Agarose-Cellulose Nanocomposite Porous Gel Microsphere

Instrument: SCG-100 protein chromatography system protein purification instrument

Chromatography column: cytiva Tricorn 10/100 Column

Mobile phase: pure water

Testing: 8 mL of agarose-cellulose nanocomposite porous gel microspheres were loaded on the abovementioned chromatography column, the flow rate started from 0.5 mL/min, and the pressure was detected. The flow rate was gradually increased every 5 minutes until the system pressure rose sharply to 3 MPa, indicating that the sample collapsed and the flow rate could not continue to increase, and the test was ended. The increasing sequence of flow rate was 0.5, 1.0, 1.5, 2.0, 3.5, 4.0, 4.5, 5.0, 6.0, 7.0, 8.0, 9.0, 10.0, 12.0, 14.0, 16.0, 18.0, . . . , 66.0 mL/min. The volumetric flow rate was converted into linear velocity: V=(60×V_v)/S, where V was the linear velocity (cm/h), V_v was the volumetric flow rate (mL/min), and S was the cross-sectional area of 0.785 cm² of the chromatography column. The flow rate and column pressure in the last stage of stable pressure before the pressure raised sharply were defined as the maximum flow rate and pressure resistance of the porous gel microspheres.

The cross-linked agarose-cellulose nanocomposite porous gel microspheres in Example 1 had an average particle size of 109 μm, a maximum flow rate of 3000 cm/h, and a pressure resistance of 0.45 MPa. The average particle size of the agarose porous gel microspheres without nanocellulose in Comparative Example 1 was 107 μm, the maximum flow rate was 1,350 cm/h, and the pressure resistance was 0.20 MPa. The difference between Example 1 and Comparative Example 1 was that in Example 1, 5% nanocellulose relative to the mass of agarose was added. It could be found that when the particle sizes of the microspheres were close, the addition of a small amount of nanocellulose greatly improved the maximum flow rate and pressure resistance.

### Example 2

Regarding an application of the agarose-cellulose nanocomposite porous gel microsphere, the cross-linked agarose-cellulose nanocomposite porous gel microsphere in

Example 1 is modified with a Ni-IDA ligand as an affinity chromatography medium, and the following steps are included:
1) Allylation modification: 10 g of agarose-cellulose nanocomposite porous gel microspheres filtered and dried in a gravity column were weighed, and 3 mL of Na₂SO₄ solution with a concentration of 2.5 mol/L, 2 mL of NaOH solution with a concentration of 30 wt %, and 25 mg NaBH₄ were added, slowly added 2.5 mL of allyl glycidyl ether under stirring at 45° C., and reacted for 16 hours.
2) Bromine water activation: Allyl-modified agarose-cellulose nanocomposite porous gel microspheres were added with 3.5 mL of purified water and 2.0 g of sodium acetate and activated by adding fresh bromine water drop by drop under stirring at room temperature until the yellow color did not fade within 1 min, and then 0.04 g of sodium formate was added to remove the remaining bromine water.
3) IDA modification and Ni loading: 10 mL of sodium iminodiacetate (IDA) solution (15 wt %, adjusted to pH=11.5 with 50% NaOH solution) was added to the brominated product, and reacted at 50° C. for 18 hours. After IDA modification, Ni was loaded in the agarose-cellulose nanocomposite porous gel microspheres with 50 mmol/L NiSO₄ solution to obtain a chromatography medium whose ligand was Ni-IDA.

### Dynamic Binding Capacity Test of Ni-IDA Chromatography Medium

Instrument: AKTA explorer 100 protein protein purification system

Chromatography column: cytiva Tricorn 5/100 Column

Column packing: 2.0 mL of Ni-IDA chromatography medium was installed in the abovementioned chromatography column, equilibrated with buffer A for 3 CV (column volume), loaded with 2 mg/mL His-tagged protein A solution, and stopped when 10% flow-through was reached. The dynamic binding capacity was calculated according to the following formula: DBC_{10%}=(V_{10%}−V₀)C₀/V₀′, where V₁₀% was the sample loading volume at 10% flow-through, V₀ was the dead volume (2.33 ml) of the detection system pipeline, and V_{c} was the packing volume (2 ml) in the column.

The composition of buffer A was 16.2 mmol/L disodium hydrogen phosphate dodecahydrate, 3.8 mmol/L sodium dihydrogen phosphate dihydrate, 20 mmol/L sodium chloride, and pH=7.4.

The composition of buffer B was 16.2 mmol/L disodium hydrogen phosphate dodecahydrate, 3.8 mmol/L sodium dihydrogen phosphate dihydrate, 20 mmol/L sodium chloride, 500 mmol/L imidazole, and pH=7.6.

After the nanocomposite porous gel microspheres were modified with Ni-IDA, the dynamic binding capacity of the protein A of the microspheres in Example 2 was 49.8 mg/mL, and the dynamic binding capacity of the microspheres in Comparative Example 2 was 42.1 mg/mL. Example 2 and Comparative Example 2 were respectively the application of Example 1 and Comparative Example 1 modified with ligands, and it could be found that the addition of nanocellulose was beneficial to increase the dynamic binding capacity.

### Example 3

The preparation of an agarose-cellulose nanocomposite porous gel microsphere includes the following steps.

The amount of nanocellulose dispersion in Example 1 was changed to 15.1 g, 80.9 g of water was added, and other conditions remained unchanged. The obtained cross-linked agarose-cellulose nanocomposite porous gel had an average particle size of 124 μm, a maximum flow rate of 2,600 cm/h, and a pressure resistance of 0.38 MPa. In Example 3, the amount of nanocellulose added was 10% relative to the mass of the agarose. Compared with the 5% added amount in Example 1, the flow rate and pressure resistance were not further improved.

### Example 4

The preparation of an agarose-cellulose nanocomposite porous gel microsphere includes the following steps.

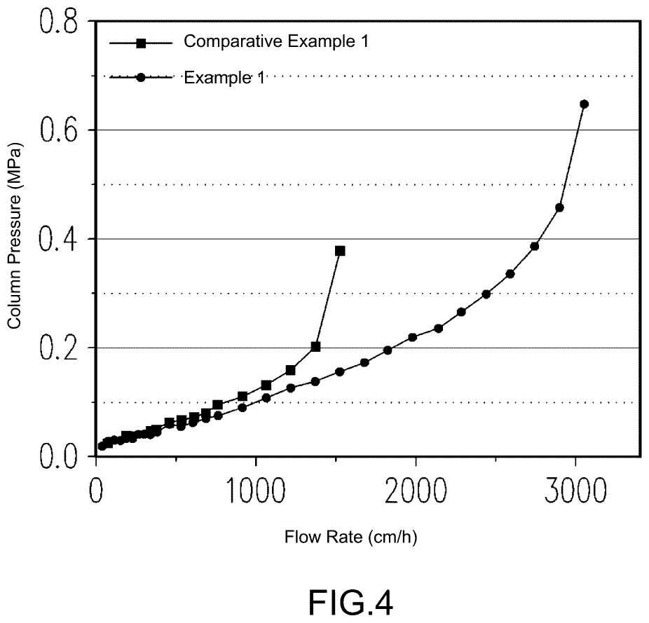

The type of nanocellulose in Example 1 was changed to CM-CNF (concentration: 0.38%), the amount of dispersion was changed to 52.6 g, 43.4 g of water was added, and other conditions remained unchanged. The obtained cross-linked agarose-cellulose nanocomposite porous gel had an average particle size of 117 μm, a maximum flow rate of 2,750 cm/h, and a pressure resistance of 0.39 MPa. The pressure/flow rate characteristic curve of Comparative Example 1 is shown in FIG. 4. It can be found that CM-CNF also has a significant enhancing effect.

### Example 5

The preparation of an agarose-cellulose nanocomposite porous gel microsphere includes the following steps.

The amount of nanocellulose in Example 4 was changed to 31.6 g, 64.4 g of water was added, and other conditions remained unchanged. The obtained cross-linked agarose-cellulose nanocomposite porous gel had an average particle size of 89 μm, a maximum flow rate of 2100 cm/h, and a pressure resistance of 0.33 MPa.

### Example 6

The preparation of an agarose-cellulose nanocomposite porous gel microsphere includes the following steps.

The amount of water added to the 52.6 g nanocellulose dispersion in Example 4 was changed to 45.4 g, the amount of agarose was changed to 2.0 g, the emulsification speed was changed to 1,000 rpm, and other conditions remained unchanged. The obtained cross-linked agarose-cellulose nanocomposite porous gel had an average particle size of 107 μm, a maximum flow rate of 450 cm/h, and a pressure resistance of 0.06 MPa.

### Example 7

The preparation and application of an agarose-cellulose nanocomposite porous gel microsphere includes the following steps.

The type of nanocellulose in Example 1 was changed to CNC (concentration: 0.50%), the amount of CNC dispersion was 8.0 g, 88.0 g of water was added, and other conditions remained unchanged. The obtained cross-linked agarose-cellulose nanocomposite porous gel had an average particle size of 109 μm and was modified with Ni-IDA by the method in Example 2. As an affinity chromatography medium, the dynamic binding capacity of protein A was 46.3 mg/mL. It could be found that different types of nanocellulose exhibited the effect of increasing the dynamic binding capacity.

### Comparative Example 1

The preparation of an agarose porous gel microsphere includes the following steps.

The amount of nanocellulose in Example 1 was changed to 0 g, 96.0 g of water was added, and other conditions remained unchanged. The obtained cross-linked agarose-cellulose nanocomposite porous gel had an average particle size of 109 μm, a maximum flow rate of 1,350 cm/h, and a pressure resistance of 0.20 MPa.

### Comparative Example 2

The application of an agarose porous gel microsphere includes the following steps.

The cross-linked agarose-cellulose nanocomposite porous gel microspheres in Example 2 were changed to the cross-linked agarose porous gel microspheres in Comparative Example 1 for Ni-IDA ligand modification. As an affinity chromatography medium, the dynamic binding capacity of protein A was 42.1 mg/mL.

### Comparative Example 3

The preparation of an agarose porous gel microsphere includes the following steps.

The amount of nanocellulose in Example 6 was changed to 0 g, 98.0 g of water was added, and other conditions remained unchanged. The obtained cross-linked agarose-cellulose nanocomposite porous gel had an average particle size of 97 μm, a maximum flow rate of 190 cm/h, and a pressure resistance of 0.05 MPa.

The above specific embodiments are used to explain the disclosure, and are only preferred embodiments of the disclosure, rather than limiting the disclosure. Any modifications, equivalent replacements, improvements, etc. made to the disclosure without departing from the spirit and protection scope of the claims of the disclosure shall fall within the protection scope of the disclosure.

What is claimed is:

1. A preparation method of an agarose-cellulose nanocomposite porous gel microsphere, comprising:
dissolving an agarose in a dispersion of a nanocellulose as a water phase,
wherein 0.01 to 10 parts by weight of the nanocellulose are dispersed in 100 parts by weight of water to form a uniform dispersion, and 0.5-20 parts by weight of the agarose is added and heated under stirring until the agarose is completely dissolved;
preparing the agarose-cellulose nanocomposite porous gel microsphere through a reversed-phase emulsification method,
wherein the water phase is poured into an oil phase heated to 50-90° C., mechanically stirred and emulsified for 10-30 minutes, a rotation speed is adjusted so that the water phase is dispersed into a droplet of a required particle size, the emulsion is cooled at a rate of 2° C. per minute to below 20° C. to gel the droplet of the water phase, and an uncrosslinked agarose-cellulose nanocomposite gel microsphere is obtained after washing,
wherein the oil phase comprises a water-immiscible organic solvent and a single or compound emulsifier with an HLB value of 3 to 8; and
cross-linking the agarose-cellulose nanocomposite porous gel microsphere,
wherein epichlorohydrin is used to cross-link agarose and cellulose under alkaline conditions to form the agarose-cellulose nanocomposite porous gel microsphere, and an amount of the epichlorohydrin is 1-20% based on a volume of the microsphere.

2. The preparation method of the agarose-cellulose nanocomposite porous gel microsphere according to claim 1, wherein
the nanocellulose is cellulose nanofibrils or cellulose nanocrystal,
the cellulose nanofibrils are cellulose nanofibrils RHEOCRYSTA or cellulose nanofibrils modified by carboxymethylation, and
the cellulose nanocrystal is obtained by hydrolysis of microcrystalline cellulose.

3. The preparation method of the agarose-cellulose nanocomposite porous gel microsphere according to claim 1, wherein
the nanocellulose is present in an aggregated state with a diameter of 2-100 nm and a length of less than 10 μm, is fibril-shaped or rod-shaped, and is not comb-shaped or fork-shaped.

4. The preparation method of the agarose-cellulose nanocomposite porous gel microsphere according to claim 1, wherein
a crystalline region is provided in the nanocellulose, and a surface of the nanocellulose has a molecular chain of cellulose or a molecular chain of cellulose derivative.

5. The preparation method of the agarose-cellulose nanocomposite porous gel microsphere according to claim 1, wherein
in dissolving an agarose in a dispersion of a nanocellulose, the agarose is 4 to 6 parts by weight, and the nanocellulose is 0.1 to 1 parts by weight.

6. The preparation method of the agarose-cellulose nanocomposite porous gel microsphere according to claim 1, wherein
in preparing the agarose-cellulose nanocomposite porous gel microsphere, the organic solvent in the oil phase is at least one of cyclohexane and liquid paraffin, and
the emulsifier in the oil phase is at least one of Span 85, Span 80 and Span 60.

7. An agarose-cellulose nanocomposite porous gel microsphere, wherein
the agarose-cellulose nanocomposite porous gel microsphere is prepared and obtained through the preparation method of the agarose-cellulose nanocomposite porous gel microsphere according to claim 1.

8. The agarose-cellulose nanocomposite porous gel microsphere according to claim 7, wherein
a mass of the nanocellulose contained in the agarose-cellulose nanocomposite porous gel microsphere accounts for 0.1-200% of a mass of the agarose, and
the agarose-cellulose nanocomposite porous gel microsphere is spherical or approximately spherical, with a diameter of 20-300 μm.

9. A chromatography medium having an agarose-cellulose nanocomposite porous gel microsphere, wherein
the chromatography medium is obtained by modifying the agarose-cellulose nanocomposite porous gel microsphere according to claim 7 with a ligand.

10. An application of the agarose-cellulose nanocomposite porous gel microsphere according to claim 7 for separating and purifying a biological macromolecule.
